(19) United States
(12) Patent Application Publication
(10) Pub. No.: US 2024/0389945 A1
(43) Pub. Date: Nov. 28, 2024

(54) SYSTEM FOR INTRAVASCULAR AND NON-INVASIVE PHYSIOLOGICAL AND VASCULAR PARAMETER SENSING

(71) Applicant: Becton, Dickinson and Company, Franklin Lakes, NJ (US)

(72) Inventors: Jonathan Karl Burkholz, Salt Lake City, UT (US); Jeffrey C. O'Bryan, Murray, UT (US)

(21) Appl. No.: 18/658,810

(22) Filed: May 8, 2024

Related U.S. Application Data
(60) Provisional application No. 63/469,237, filed on May 26, 2023.

(51) Int. Cl. A61B 5/00; A61B 5/0205

(52) U.S. Cl. CPC A61B 5/6852; A61B 5/0205; A61B 5/681; A61B 5/7264; A61B 2560/0443

(57) ABSTRACT

The present disclosure relates generally to systems for intravascular and non-invasive physiological and vascular parameter sensing. A system may include a wearable device, such as a watch, and a probe unit having a probe that may be inserted into the vasculature or subcutaneously to sense physiological and/or vascular parameters and send such parameters to the wearable device. The probe unit could be coupled directly to the wearable device or wirelessly coupled. A system could also include a parameter sensing patch that could sense additional physiological and/or vascular parameters. The physiological and vascular parameters can enable clinicians to make more informed decisions and can enhance tracking improvements or deteriorations in patient health status. The use of the physiological and vascular parameters may be particularly beneficial when a patient is receiving medication or fluid interventions or treatment plans.

### RELATED APPLICATIONS

This application claims the benefit of U.S. Provisional Patent Application No. 63/469,237, which was filed on May 26, 2023, which is incorporated herein in its entirety.

### BACKGROUND

Health apps and wearable devices capable of measuring physiological parameters noninvasively have become an incredibly popular and increasingly important way to empower consumers and users with important health tracking and monitored health information to better manage their activities, lifestyle, and certain chronic health conditions. In spite of the advances in this technology, certain physiological parameters may only be measured, or may only be measured with enough precision to inform clinical decision making, from within the vasculature (venous or arterial), subcutaneously or via smart implantable devices.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to systems for intravascular and non-invasive physiological and vascular parameter sensing. A system may include a wearable device, such as a watch, and a probe unit having a probe that may be inserted into the vasculature or subcutaneously to sense physiological and/or vascular parameters and send such parameters to the wearable device. The probe unit could be coupled directly to the wearable device or wirelessly coupled. A system could also include a parameter sensing patch that could sense additional physiological and/or vascular parameters. The physiological and vascular parameters can enable clinicians to make more informed decisions and can enhance tracking improvements or deteriorations in patient health status. The use of the physiological and vascular parameters may be particularly beneficial when a patient is receiving medication or fluid interventions or treatment plans.

In some embodiments of the present disclosure, a system for intravascular and non-invasive physiological and vascular parameter sensing may include a wearable device and a probe unit that is configured to couple to the wearable device. The probe unit may have a probe that is configured to be inserted into a vasculature or subcutaneously to sense one or more physiological or vascular parameters and to deliver the one or more physiological or vascular parameters to the wearable device.

In some embodiments, the probe unit may physically couple to the wearable device.

In some embodiments, the probe unit may physically couple to a band of the wearable device.

In some embodiments, the probe unit may physically couple to a computing unit of the wearable device.

In some embodiments, the probe unit may include a base unit from which the probe extends and an interface that is connected to the base unit, the interface physically coupling to the wearable device.

In some embodiments, the probe unit may include a base unit that couples wirelessly to a computing unit of the wearable device.

In some embodiments, the probe may include one or more sensors that sense the one or more physiological or vascular parameters.

In some embodiments, the probe unit may include a platform.

In some embodiments, the one or more physiological or vascular parameters may include one or more of pH, lactate, glucose, arterial or venous core temperature, blood pressure, oxygenation levels, presence of microbes, blood chemistry, blood gases, or electrolytes.

In some embodiments, the wearable device may include a computing unit that displays the one or more physiological or vascular parameters.

In some embodiments, the wearable device may be a watch, and the probe unit may couple to a band of the watch.

In some embodiments, the system may include a parameter sensing patch that is configured to communicate with the probe unit.

In some embodiments, the probe unit may be configured to insert into a vascular access device to position the probe in the vasculature or subcutaneously.

In some embodiments, the system may include a server that is configured to receive the one or more physiological or vascular parameters from the wearable device.

In some embodiments, the server may be configured to process the one or more physiological or vascular parameters using an artificial intelligence algorithm to detect or predict an occurrence of a health condition.

In some embodiments of the present disclosure, a system for intravascular and non-invasive physiological and vascular parameter sensing may include a watch and a probe unit. The probe unit may have a base unit from which a probe extends. The base unit may be configured to couple to the watch. The probe may be configured to sense one or more physiological or vascular parameters when positioned in an individual's vasculature or subcutaneously while the individual wears the watch.

In some embodiments, the probe unit may include an interface that inserts into a socket of the watch.

In some embodiments, the socket may be formed on a band of the watch or in a computing unit of the watch.

In some embodiments, the probe unit may wirelessly couple to the watch.

In some embodiments of the present disclosure, a system for intravascular and non-invasive physiological and vascular parameter sensing may include a watch having a band and a probe unit having a base unit, a probe having one or more sensors, and an interface. The one or more sensors may generate one or more physiological or vascular parameters when the probe is inserted into an individual's vasculature or subcutaneously. The interface may insert into a socket of the band to form an electrical connection between a computing unit of the watch and the base unit for transferring the one or more physiological or vascular parameters.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1 provides an example of a system for intravascular and non-invasive physiological and vascular parameter sensing that is configured in accordance with one or more embodiments of the present disclosure;

FIG. 2 provides another example of a system for intravascular and non-invasive physiological and vascular parameter sensing that is configured in accordance with one or more embodiments of the present disclosure;

FIG. 3 provides another example of a system for intravascular and non-invasive physiological and vascular parameter sensing that is configured in accordance with one or more embodiments of the present disclosure;

FIG. 4 provides another example of a system for intravascular and non-invasive physiological and vascular parameter sensing that is configured in accordance with one or more embodiments of the present disclosure;

FIGS. 4A and 4B illustrate variations of the system of FIG. 4;

FIG. 5 provides an example of how a probe unit of a system for intravascular and non-invasive physiological and vascular parameter sensing can be inserted into an individual's vasculature;

FIG. 6 provides another example of a system for intravascular and non-invasive physiological and vascular parameter sensing that is configured in accordance with one or more embodiments of the present disclosure;

FIG. 7 provides another example of a system for intravascular and non-invasive physiological and vascular parameter sensing that is configured in accordance with one or more embodiments of the present disclosure;

FIG. 8 provides another example of a system for intravascular and non-invasive physiological and vascular parameter sensing that is configured in accordance with one or more embodiments of the present disclosure; and

FIG. 9 provides another example of a system for intravascular and non-invasive physiological and vascular parameter sensing that is configured in accordance with one or more embodiments of the present disclosure.

### DESCRIPTION OF EMBODIMENTS

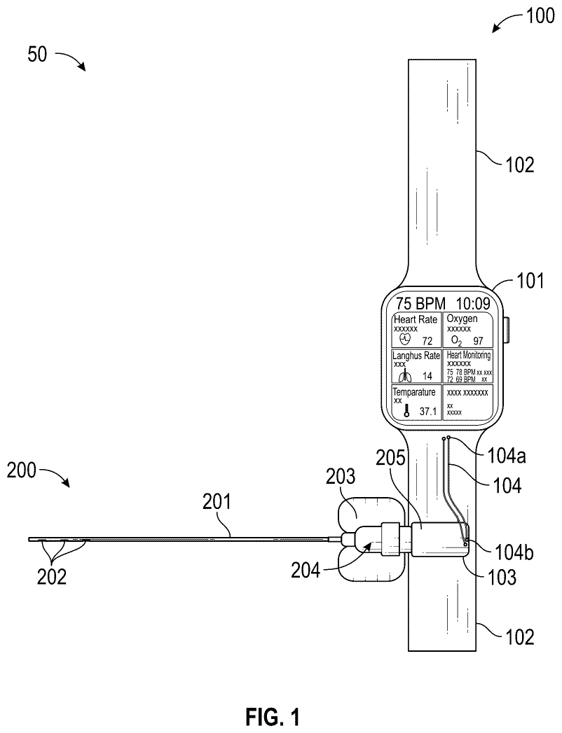

FIG. 1 provides an example of a system 50 for intravascular and non-invasive physiological and vascular parameter sensing that is configured in accordance with one or more embodiments of the present disclosure. System 50 includes a wearable device 100 and a probe unit 200. In some embodiments, such as the depicted embodiments, wearable device 100 can be in the form of a watch (or bracelet) that includes a computing unit 101 and bands 102 to enable wearable device 100 to be worn around the wrist.

Probe unit 200 can include a probe 201, one or more sensors 202, a platform 203, a base unit 204, and an interface 205. Probe 201 is configured to be inserted into an individual's vasculature or subcutaneously. In some embodiments, probe 201 may include a core wire, such as a nitinol wire running along the length of probe 201, for structure and durability during advancement. In some embodiments, probe 201 may include an atraumatic tip. In some embodiments, probe 201 could include a fluid path to enable blood draw or aspiration via probe unit 200.

In some embodiments, probe 201 could include a coating to improve performance or reduce the risk of complications such as development of thrombus or probe related blood stream infection. These coatings may include silicon lube with or without an antimicrobial additive, such as CH (x). In some embodiments, probe 201 may be coated with anti-thrombogenic or anti-microbial coatings or polymer additives.

Sensors 202 can be positioned on/in probe 201 where they will be in contact with blood, tissue, etc. for sensing one or more physiological or vascular parameters below the surface of the skin. In some embodiments, base unit 204 can include circuitry for controlling and/or communicating with sensors 202 (e.g., via optical fibers or wires). Platform 203 can support base unit 204 when platform 203 is placed on the surface of the skin.

Interface 205 is a physical and electrical interface for coupling probe unit 200 to wearable device 100. For example, in FIG. 1, a band 102 of wearable device 100 includes a socket 103 for receiving interface 205. One or more leads 104 can be integrated into band 102 and may form contacts 104a for connecting leads 104 to corresponding contacts on computing unit 101 and contacts 104b for connecting leads 104 to corresponding contacts on interface 205. In other words, when interface 205 is inserted into socket 103, an electrical connection will be established between computing unit 101 and base unit 204 to thereby enable computing unit 101 to obtain data generated by sensors 202 indicative of the one or more physiological or vascular parameters. In some embodiments, leads 104 may also provide power to base unit 204. However, in other embodiments, base unit 204 may be independently powered (e.g., via a battery).

Socket 103 can be positioned and oriented on band 102 to enable probe 201 to be inserted into an individual's vasculature or subcutaneously while interface 205 is inserted into socket 103. For example, in the configuration depicted in FIG. 1, probe 201 may be inserted into a vein located on the underside of the individual's wrist. Once probe 201 is positioned properly, a securement dressing (not shown) could be placed over platform 203 to secure probe 201 in place.

In some embodiments, sensors 202 can be configured to sense one or more blood-based parameters such as pH, lactate, glucose, arterial or venous core temperature, blood pressure, oxygenation levels, presence of microbes, blood chemistry, blood gases, electrolytes, etc. In some embodiments, computing unit 101 can be configured to display such parameters (e.g., via an app) and/or to transmit such parameters to another system (e.g., to a server). In some embodiments, computing unit 101 may also be configured to display additional parameters such as heart rate, irregular heart rhythm, ECG, low cardio fitness, blood oxygen levels, fall detection, etc.

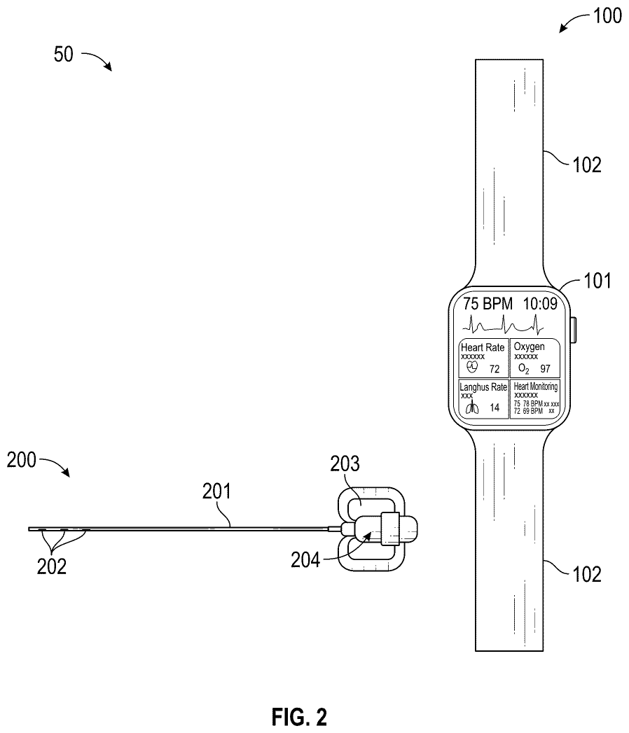

FIG. 2 provides another example of a system 50 for intravascular and non-invasive physiological and vascular parameter sensing that is configured in accordance with one or more embodiments of the present disclosure. System 50 shown in FIG. 2 is generally the same as in FIG. 1 except that base unit 204 is configured to communicate wirelessly with computing unit 101. As such, probe unit 200 does not include interface 205 and band 102 does not include socket 103 or leads 104.

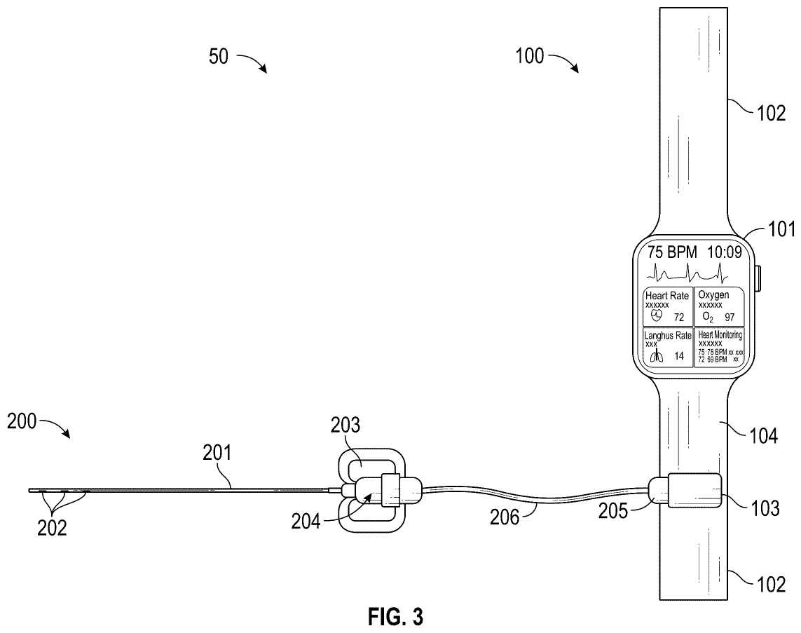

FIG. 3 provides another example of a system 50 for intravascular and non-invasive physiological and vascular parameter sensing that is configured in accordance with one or more embodiments of the present disclosure. System 50 shown in FIG. 3 is generally the same as in FIG. 1 except that base unit 204 is connected to interface 205 via a tether 206. This configuration provides greater flexibility in the placement of probe 201.

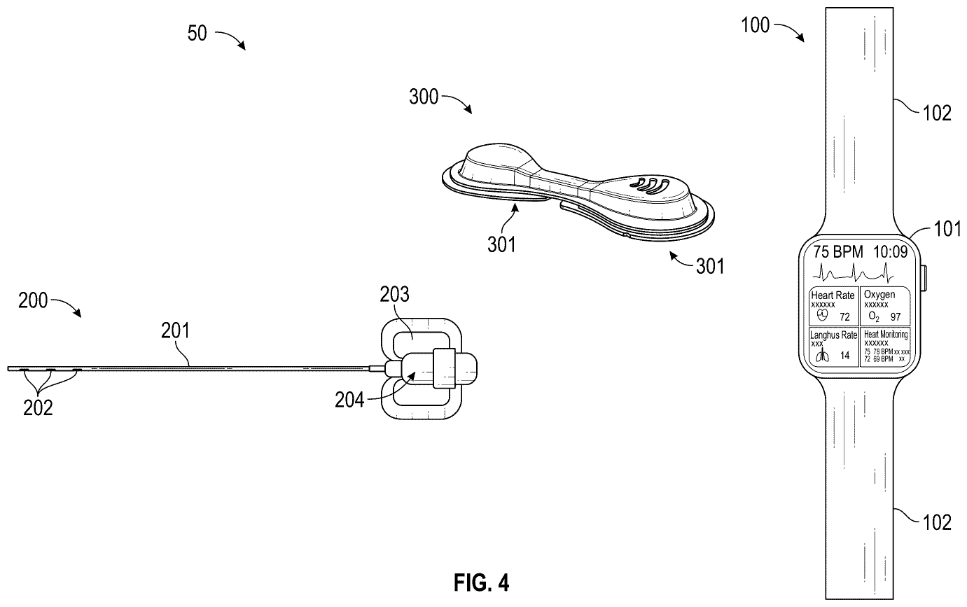

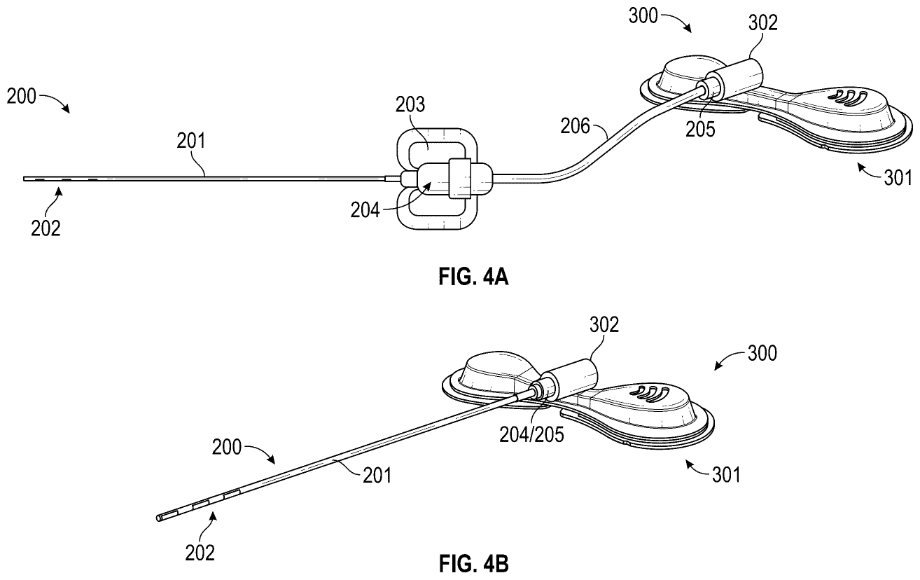

FIG. 4 provides another example of a system 50 for intravascular and non-invasive physiological and vascular parameter sensing that is configured in accordance with one or more embodiments of the present disclosure. System 50 shown in FIG. 4 is similar to system 50 of FIG. 2 but with the addition of a parameter sensing patch 300. Parameter sensing patch 300 can have a base 301 that is configured to be adhered to an individual's skin and can be configured to sense additional physiological and vascular parameters such as vitals. In some embodiments, both base unit 204 and parameter sensing patch 300 can be configured to wirelessly transmit physiological and vascular parameters to computing unit 101. In other embodiments, base unit 204 may be configured to wirelessly transmit physiological and vascular parameters to parameter sensing patch 300 which can then relay such parameters and possibly additional parameters to computing unit 101. In some embodiments, system 50 may only include probe unit 200 and parameter sensing patch 300. In other words, in embodiments that include parameter sensing patch 300, wearable device 100 may not be used.

FIG. 4A shows a variation in which parameter sensing patch 300 includes a socket 302 for receiving interface 205 that is connected to base unit 204 via tether 206. FIG. 4B shows a variation in which interface 205 is connected directly to base unit 204 and inserts into socket 302.

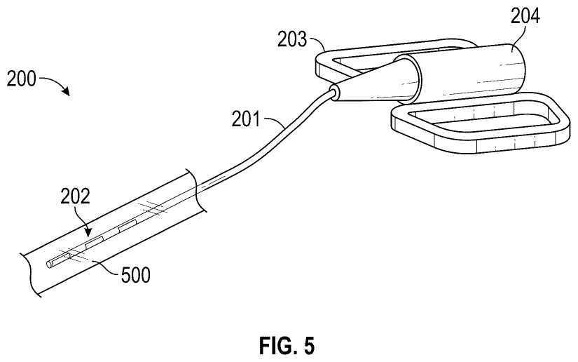

FIG. 5 provides an example of how probe unit 200 may be used to sense physiological and vascular parameters from within an individual's vasculature 500. Probe 201 can be inserted into the vasculature 500 (or subcutaneously) in any suitable way such as via direct placement or via a catheter. With probe 201 inserted, platform 203 can be secured to the individual's skin such as via an adhesive on the bottom of platform 203 and/or via a securement dressing.

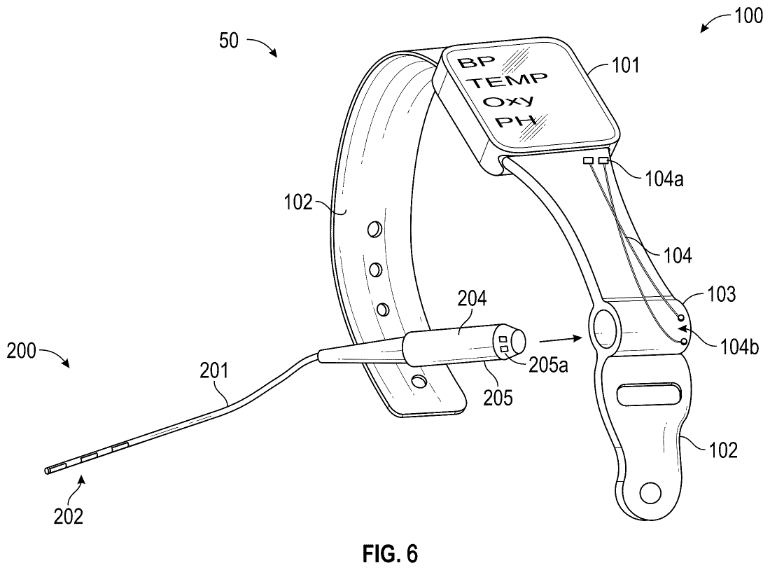

FIG. 6 provides another example of a system 50 for intravascular and non-invasive physiological and vascular parameter sensing that is configured in accordance with one or more embodiments of the present disclosure. System 50 shown in FIG. 6 is generally the same as in FIG. 1. However, probe unit 200 does not include a platform. Instead, socket 103 can function to support base unit 204. FIG. 6 also shows how interface 205 can include contacts 205a which can make an electrical connection with contacts 104b when interface 205 is inserted into socket 103. In some embodiments, socket 103 and interface 205 can include guides, magnets, or other mechanism to ensure that contacts 205a will align with contacts 104b.

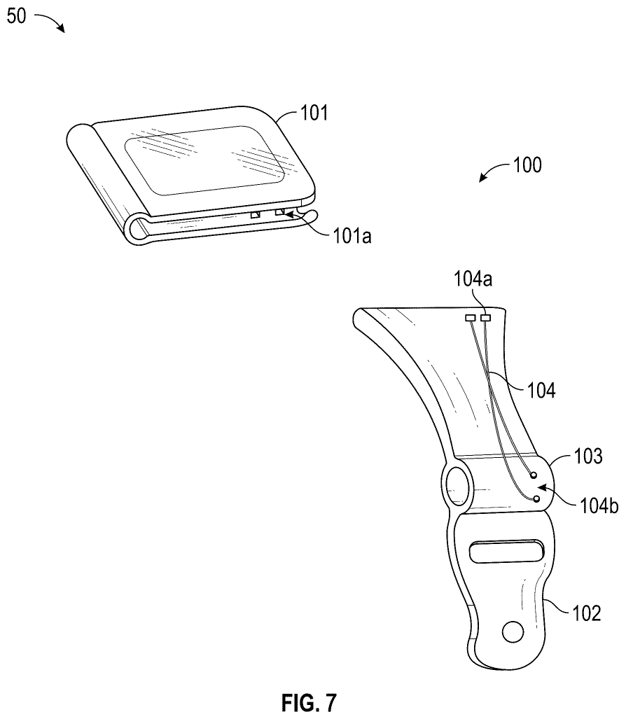

FIG. 7 provides another example of a system 50 for intravascular and non-invasive physiological and vascular parameter sensing that is configured in accordance with one or more embodiments of the present disclosure. In FIG. 7, computing unit 101 has a different configuration than in previous figures.

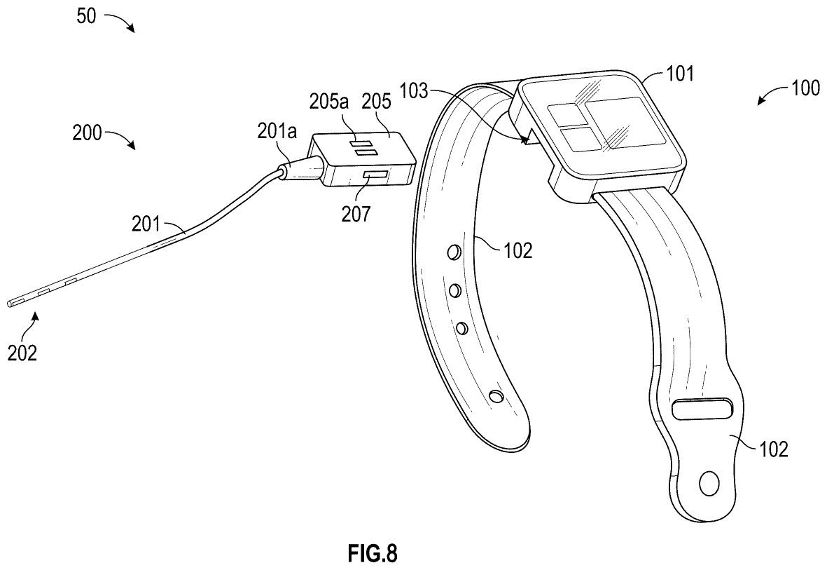

FIG. 8 provides another example of a system 50 for intravascular and non-invasive physiological and vascular parameter sensing that is configured in accordance with one or more embodiments of the present disclosure. In FIG. 8, socket 103 is formed in computing unit 101 rather than in band 102. Interface 205 also include guides 207 (e.g., indents or protrusions) for securing interface 205 in socket 103. A strain relief 201a may also be formed at the base of probe 201.

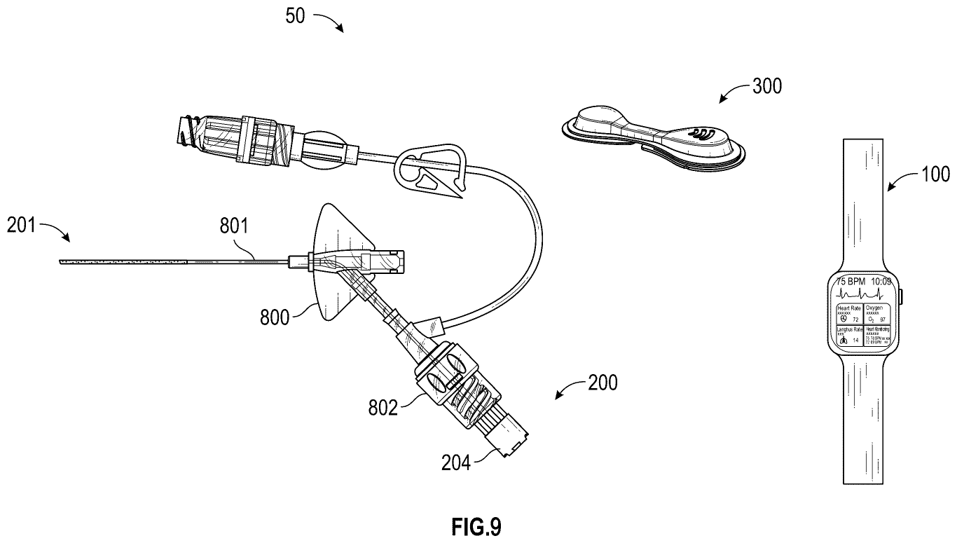

FIG. 9 provides another example of a system 50 for intravascular and non-invasive physiological and vascular parameter sensing that is configured in accordance with one or more embodiments of the present disclosure. In FIG. 9, probe unit 200 is shown as being inserted into an individual's vasculature via a vascular access device 800. In particular, probe 201 can be inserted through a port 802 of vascular access device 800 and out through a catheter 801. The length of probe 201 can be configured to cause it to extend from catheter 801 when base unit 204 is inserted into port 802. In these embodiments, system 50 may or may not include parameter sensing patch 300. Also, in these embodiments, probe unit 200 could include tether 206 for connecting base unit 204 to computing device 100 or parameter sensing patch 300.

The physiological and vascular parameters that can be obtained via system 50 can enable clinicians to make more informed decisions and can enhance tracking improvements or deteriorations in patient health status. The use of the physiological and vascular parameters may be particularly beneficial when a patient is receiving medication or fluid interventions or treatment plans.

As suggested above, in some embodiments, system 50 may include a server system (e.g., a cloud-based system) that can receive, process and store the physiological and vascular parameters (e.g., as part of a patient's electronic medical record). Such a server system can enable the physiological and vascular parameters to be displayed to clinicians that may be monitoring a patient (e.g., at a nurse station). In some embodiments, the server system may process the physiological and vascular parameters and then relay them, or data derived therefrom, to an app on computing unit 101 for display. In some embodiments, the server system may include an artificial intelligence engine that can process the physiological and vascular parameters to automatically detect or predict the occurrence of a condition.

In some embodiments, computing unit 101 or another computing device could be configured to output an alert based on the physiological and vascular parameters. For example, an app or dedicated user interface components on computing unit 101 could be configured to output visual, audible, tactile, or digital alerts or indicators. In some embodiments, computing unit 101 or other devices (e.g., an infusion pump, a vital sign monitor, an arterial monitor, an ultrasound system visual display, a smart phone, a tablet, etc.) could respond to such alerts or indicators, or directly to the physiological and vascular parameters, to capture data or images for storage with the physiological and vascular parameters.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed:

1. A system for intravascular and non-invasive physiological and vascular parameter sensing comprising:
a wearable device; and
a probe unit that is configured to couple to the wearable device, the probe unit having a probe that is configured to be inserted into a vasculature or subcutaneously to sense one or more physiological or vascular parameters and to deliver the one or more physiological or vascular parameters to the wearable device.

2. The system of claim 1, wherein the probe unit physically couples to the wearable device.

3. The system of claim 1, wherein the probe unit physically couples to a band of the wearable device.

4. The system of claim 1, wherein the probe unit physically couples to a computing unit of the wearable device.

5. The system of claim 1, wherein the probe unit includes a base unit from which the probe extends and an interface that is connected to the base unit, the interface physically coupling to the wearable device.

6. The system of claim 1, wherein the probe unit includes a base unit that couples wirelessly to a computing unit of the wearable device.

7. The system of claim 1, wherein the probe includes one or more sensors that sense the one or more physiological or vascular parameters.

8. The system of claim 1, wherein the probe unit includes a platform.

9. The system of claim 1, wherein the one or more physiological or vascular parameters include one or more of pH, lactate, glucose, arterial or venous core temperature, blood pressure, oxygenation levels, presence of microbes, blood chemistry, blood gases, or electrolytes.

10. The system of claim 9, wherein the wearable device includes a computing unit that displays the one or more physiological or vascular parameters.

11. The system of claim 1, wherein the wearable device is a watch, and wherein the probe unit couples to a band of the watch.

12. The system of claim 1, further comprising:
a parameter sensing patch that is configured to communicate with the probe unit.

13. The system of claim 1, wherein the probe unit is configured to insert into a vascular access device to position the probe in the vasculature or subcutaneously.

14. The system of claim 1, further comprising:
a server that is configured to receive the one or more physiological or vascular parameters from the wearable device.

15. The system of claim 14, wherein the server is configured to process the one or more physiological or vascular parameters using an artificial intelligence algorithm to detect or predict an occurrence of a health condition.

16. A system for intravascular and non-invasive physiological and vascular parameter sensing comprising:
a watch; and
a probe unit having a base unit from which a probe extends, the base unit being configured to couple to the watch, the probe being configured to sense one or more physiological or vascular parameters when positioned in an individual's vasculature or subcutaneously while the individual wears the watch.

17. The system of claim 16, wherein the probe unit includes an interface that inserts into a socket of the watch.

18. The system of claim 17, wherein the socket is formed on a band of the watch or in a computing unit of the watch.

19. The system of claim 16, wherein the probe unit wirelessly couples to the watch.

20. A system for intravascular and non-invasive physiological and vascular parameter sensing comprising:
a watch having a band; and
a probe unit having a base unit, a probe having one or more sensors, and an interface, wherein the one or more sensors generate one or more physiological or vascular parameters when the probe is inserted into an individual's vasculature or subcutaneously, and wherein the interface inserts into a socket of the band to form an electrical connection between a computing unit of the watch and the base unit for transferring the one or more physiological or vascular parameters.
